(19) United States
(12) Patent Application Publication
(10) Pub. No.: US 2017/0312376 A1
(43) Pub. Date: Nov. 2, 2017

(54) HYDROGEN PEROXIDE PLASMA IONIZATION GENERATOR DEVICE HAVING A DOUBLE-JET NOZZLE

(71) Applicant: TAEWANG SCIENCE CO., LTD., SEOUL (KR)

(72) Inventor: CHAN WON BAE, SEOUL (KR)

(73) Assignee: TAEWANG SCIENCE CO., LTD., Seoul (KR)

(21) Appl. No.: 15/524,117

(22) PCT Filed: Nov. 5, 2015
(86) PCT No.: PCT/KR2015/011866

§ 371 Date: May 3, 2017

(30) Foreign Application Priority Data
Nov. 5, 2014 (KR) 10-2014-0152547

(51) Int. Cl. A61L 2/14; A61L 2/20; H05H 1/48

(52) U.S. Cl. CPC A61L 2/14; H05H 1/48; A61L 2/208; A61L 2202/11; H05H 2245/123; H01J 2237/327

(57) ABSTRACT

According to the hydrogen peroxide plasma ionization generator device having a double-jet nozzle suggested by the present invention, the double jet nozzle forming an air passage is configured to construct the hydrogen peroxide plasma ionization generator device such that the hydrogen peroxide and ion particles are sprayed finer than that of a conventional single-jet nozzle.

### TECHNICAL FIELD

The present invention relates to a hydrogen peroxide plasma ionization generator device having a double-jet nozzle, and more specifically relates to a hydrogen peroxide plasma ionization generator device having a double-jet nozzle of which an air inflow passage is formed into a double-jet nozzle structure capable of spraying the hydrogen peroxide and ion particles finer than a conventional single-jet nozzle.

### BACKGROUND ART

In general, an inside laboratory and a manufacturing room at a GMP (Good Manufacturing Place), an animal feed facility, a creature safety such as BSL-3 (Biological Safety Level-3) or an aseptic preparation and manufacturing facility need a management for pure sterilization. The sterilization means a high level treatment in an aspect that every species of living microbes are perfectly removed by physical and chemical reaction different from cleaning or disinfection. EO (Ethylene Oxide) gas, steam, hydrogen peroxide, plasma, etc. are used for a sterilization method.

A sterilization using EO gas has high explosion danger in itself and has been introduced as hereditary toxic chemical causing a mutation. A steam sterilizer is evaluated at one of safety methods to obtain a predetermined level of sterilizing power. The steam sterilizer has advantages of toxicity free, relatively low cost and fast sterilization, but can be used only to a medical instrument without side effects even if it is exposed at humidity and high temperature. It is disclosed that hydrogen peroxide, ozone and a plasma generator are aptly combined to use. For example, there are disclosed that a method of providing hydrogen peroxide in a sterilizing chamber and generating plasma to perform the sterilization, a method of providing plasma and sterilizing agent in a sterilizing chamber simultaneously, a method of providing oxygen in a sterilizing chamber, generating plasma and converting into ozone to perform sterilization, and a method of providing hydrogen peroxide and ozone simultaneously to sterilize a target being sterilized in a sterilizing chamber.

At a conventional sterilizing apparatus using hydrogen peroxide and plasma, a vapor molecule is not permeated through a fine space because of a big vapor molecule (1˜5 micron) during sterilization and a perfect sterilization may not be achieved for an entire room inside. Thus, the conventional sterilizing apparatus cannot sterilize the room inside and the fine space between instruments to have disadvantage of low sterilizing efficiency. Korea Patent No. 10-0782040 discloses a method of sterilization using hydrogen peroxide and ozone as a prior art, and Korea Utility No. 20-0438487 discloses a sterilizing apparatus having a plurality of sterilizing chambers as a prior art.

### DISCLOSURE

### Technical Problem

The present invention is suggested to solve the above problems of conventional methods and provides a hydrogen peroxide plasma ionization generator device having a double-jet nozzle of which an air inflow passage is formed into a double-jet nozzle structure capable of spraying the hydrogen peroxide and ion particles finer than a conventional single-jet nozzle.

The present invention also provides a hydrogen peroxide plasma ionization generator device having a double-jet nozzle capable of further improving proper space permeation and mobility of molecular structure which is sterilizing and removing efficiently microbes, bacteria and mold at a room inside and a fine space between instruments, by configuring the double-jet nozzle to spray finer particle size than the conventional single-jet nozzle.

In addition, the present invention further provides a hydrogen peroxide plasma ionization generator device having a double-jet nozzle to generate a vapor molecular structure with a finer particle than hydrogen peroxide and ion vapor particle generated by the conventional single-jet nozzle, to improve restraint on condensation occurred during sterilization and diffusion ability for permeating in the fine space where the vapor molecule was not permeated, thereby improving sterilizing effect at the room inside or the fine space between instruments.

### Technical Solution

In order to solve the technical problem, a hydrogen peroxide plasma ionization generator device having a double-jet nozzle according to an aspect of the present invention is a hydrogen peroxide plasma ionization generator device having a double-jet nozzle including a hydrogen peroxide container and supplier containing and supplying liquid hydrogen peroxide; an air supplier with an air pump receiving external air and supplying pressurized air; a spray apparatus with a double-jet nozzle including a first jet nozzle spraying the liquid hydrogen peroxide supplied from the hydrogen peroxide container and supplier at vapor form and a second jet nozzle spraying the vapor of the hydrogen peroxide sprayed through the first jet nozzle by adding the air supplied from the air supplier; a plasma generator device installed near the second jet nozzle of the spray apparatus and generating plasma discharge to ionize the hydrogen peroxide sprayed at the vapor form into ROS (Reactive Oxygen Species) and to diffuse the ROS; and a power supplier supplying power to an electrode of the plasma generator device to generate plasma discharge at the plasma generator device.

Preferably, the hydrogen peroxide container and supplier contains or supplies liquid hydrogen peroxide. The liquid hydrogen peroxide is 7.5% low concentration hydrogen peroxide which is safe in normal exposure.

Preferably, the spray apparatus may be composed of the double-jet nozzle disposed at the center of the spray apparatus body of which the first jet nozzle sprays the liquid hydrogen peroxide supplied from the hydrogen peroxide container and supplier and the second jet nozzle smaller than the first jet nozzle in diameter is extended from the first jet nozzle; an air passage formed to surround the double-jet nozzle with disposing the double-jet nozzle at the center; and a mist spray hole formed at an end of the spray apparatus body to expose the second jet nozzle of the double jet nozzle and the air passage.

Preferably, the spray apparatus may be composed of the double-jet nozzle disposed at the center of the spray apparatus body of which the first jet nozzle sprays the liquid hydrogen peroxide supplied from the hydrogen peroxide container and supplier and the second jet nozzle holds the first jet nozzle in the inside, receives air supplied from the air supplier and spray the air with the liquid hydrogen peroxide; an air passage covering the double-jet nozzle with disposing the double-jet nozzle in the inside; and a mist spray hole formed at an end of the spray apparatus body to expose the second jet nozzle of the double jet nozzle and the air passage.

Preferably, the spray apparatus may be configured such that the hydrogen peroxide and ion sprayed through the double-jet nozzle has 0.3˜3 micron in size.

Further preferably, the plasma generator device may generate plasma discharge of 17,000 volt such that vapor of the hydrogen peroxide sprayed through the first and second jet nozzles of the spray apparatus is ionized into ROS (Reactive Oxygen Species) and diffused rapidly.

### Advantageous Effects

According to the hydrogen peroxide plasma ionization generator device having a double-jet nozzle suggested by the present invention, the double jet nozzle forming the air passage is configured to construct the hydrogen peroxide plasma ionization generator device such that the hydrogen peroxide and ion particles are sprayed finer than that of a conventional single-jet nozzle.

The present invention also provides a hydrogen peroxide plasma ionization generator device having a double-jet nozzle capable of further improving proper space permeation and mobility of molecular structure which is sterilizing and removing efficiently microbes, bacteria and mold at a room inside and a fine space between instruments, by constructing the double-jet nozzle to spray finer particle size than the conventional single-jet nozzle.

In addition, the present invention generates a vapor molecular structure with a finer particle than hydrogen peroxide and ion vapor particle generated by the conventional single-jet nozzle such that the restraint on condensation frequently occurred during sterilization and the diffusion ability for permeating in the fine space where the vapor molecule was not permeated are improved, thereby improving sterilizing effect at the room inside or the fine space between instruments.

### DESCRIPTION OF DRAWINGS

FIG. 1 is a function block diagram illustrating a hydrogen peroxide plasma generator device with a double jet nozzle according to an embodiment of the present invention.

FIG. 2 is a drawing illustrating a spray apparatus of a hydrogen peroxide plasma generator device with a double jet nozzle according to an embodiment of the present invention.

FIG. 3 is a drawing illustrating a spray apparatus of a hydrogen peroxide plasma generator device with a double jet nozzle according to another embodiment of the present invention.

FIG. 4 is a perspective view illustrating the external appearance of the spray apparatus of a hydrogen peroxide plasma generator device with a double jet nozzle according to an embodiment of the present invention.

FIG. 5 is a drawing illustrating an operation state of passing vapor through a plasma generator device in a hydrogen peroxide plasma generator device with a double jet nozzle according to an embodiment of the present invention.

100: A hydrogen peroxide plasma generator device according to an embodiment of the present

110: A hydrogen peroxide container and supplier
120: An air supplier
121: An air pump
130: A spray apparatus
130a: A spray apparatus body
131: A first jet nozzle
132: A second jet nozzle
133: A double jet nozzle
134: An air passage
135: A mist spray hole
140: A plasma generator device
141: An electrode
150: A power supplier

### MODE FOR INVENTION

A preferable embodiment will now be described more fully hereinafter with reference to the accompanying drawings in order to implement easily the present invention by those skilled in the art. In describing the preferable embodiment of the present invention, however, a description will be abbreviated when a disclosed function or a description for component make a scope of the invention ambiguously. The same symbol will be used to elements implementing similar function and operation in entire drawings. If there is referred that a part is connected to another part, it includes a case of ‘connecting directly’ with each other by interposing another device as well as a case of ‘connecting directly’ with each other. The ‘including’ a component means that another component is not excluded but included, if special opposite statement is not described.

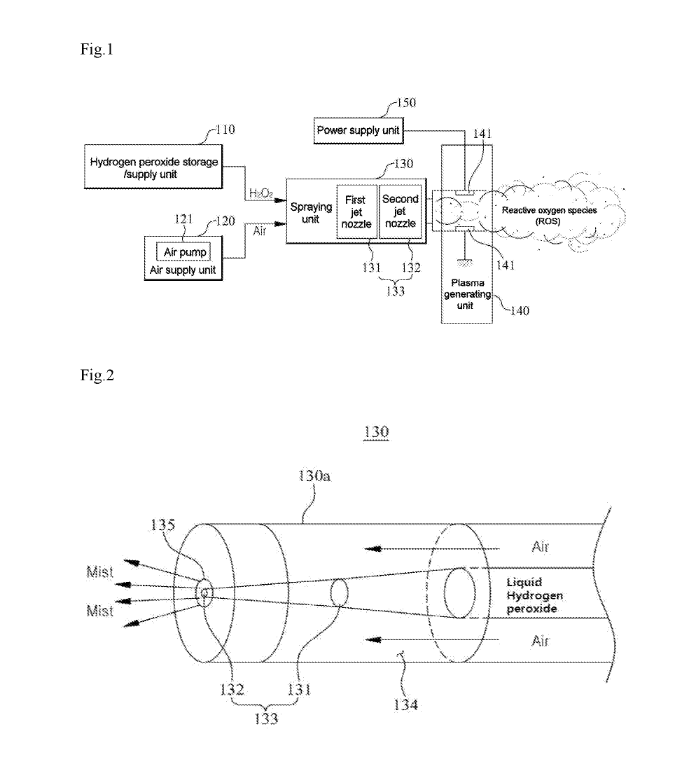

FIG. 1 is a function block diagram illustrating a hydrogen peroxide plasma generator device with a double jet nozzle according to an embodiment of the present invention. As shown in FIG. 1, a hydrogen peroxide plasma generator device 100 having a double jet nozzle according to an embodiment of the present invention may be composed of a hydrogen peroxide container and supplier 110, an air supplier 120, a plasma generator device 140 and a power supplier 150.

The hydrogen peroxide container and supplier 110 is a part for containing and supplying liquid hydrogen peroxide. The hydrogen peroxide container and supplier 110 contains and supplies the liquid hydrogen peroxide as a cleaning solution for sterilizing an inside laboratory and a manufacturing room at a GMP (Good Manufacturing Place), an animal feed facility, a creature safety such as BSL-3 (Biological Safety Level-3) or an aseptic preparation and manufacturing facility. The liquid hydrogen peroxide is a low concentration hydrogen peroxide of 7.5% which is safe in normal exposure. The low concentration hydrogen peroxide of 7.5% is similar with concentration of drug which is sold in a pharmacy without a prescription, is safe in normal exposure and has no danger in healthy even if it is exposed normally. Note that, a hydrogen peroxide over 8% in concentration is classified into a dangerous substance.

The air 120 is a part with air pump 121 for receiving outer air and supplying pressurized air to a spray apparatus 130. The air supplier 120 supplies pressurized air to a double jet nozzle 133 composed in the spray apparatus 130, get hydrogen peroxide and ion particles to be sprayed finer than that of the conventional single jet nozzle in size.

The spray apparatus 130 is configured in a double jet nozzle 133 of a first jet nozzle 131 for spraying vapor shaped liquid hydrogen peroxide supplied from the hydrogen peroxide container and supplier 110 and a second jet nozzle 132 spraying the vapor of the hydrogen peroxide from the first jet nozzle 131 with adding air supplied from the air supplier 110. The spray apparatus 130 may be configured in double wall concept of the double jet nozzle 133 including the first jet nozzle 131 and the second jet nozzle 132. The spray apparatus 130 may be configured such that the hydrogen peroxide and ion sprayed through the double-jet nozzle has 0.3˜3 micron in size. Thus, the particle size is finer than hydrogen peroxide and ion particle at the single jet nozzle which is 1˜5 micron in particle size.

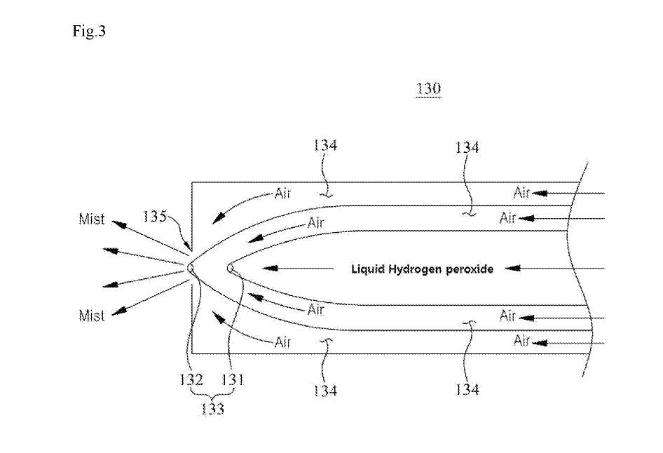

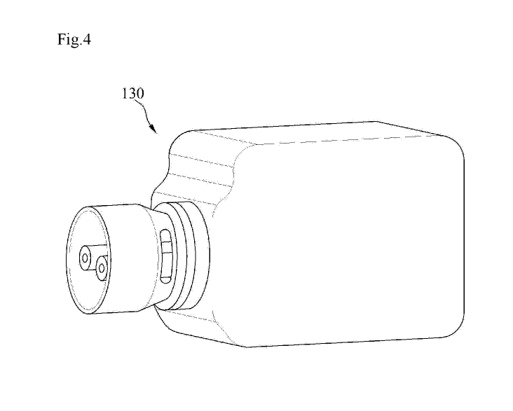

Referring to FIG. 2 and FIG. 3, the spray apparatus 130 will be specifically described.

The plasma generator device 140 is installed near the second jet nozzle of the spray apparatus and is a part for generating plasma discharge to ionize the hydrogen peroxide sprayed at the vapor form into ROS (Reactive Oxygen Species) and to diffuse the ROS. The plasma generator device 140 generates plasma discharge of 17,000 volt such that vapor of the hydrogen peroxide sprayed through the first and second jet nozzles of the spray apparatus is ionized into ROS (Reactive Oxygen Species) to diffuse rapidly in a room. The plasma generator device 140 generates ionized hydrogen peroxide molecule by the plasma discharge generated at an electrode 141, generates strong positive charge field of mesh shape, and moves in a room for sterilizing through an energy field. Ionized ROS are ionized into OH⁻, H⁺, O²⁻ to treat sterilization in the room, and a non-ionized particle is disintegrated or disappeared in the air. A hydroxide ion (OH⁻, Hydroxyl Radical) in the ROS ionized from the hydrogen peroxide is directly participated in sterilization, and a reactive oxygen (O²⁻, Radical Oxygen) sterilizes cellular composing substance such as lipid, protein, DNA, etc., simultaneously.

The power supplier 150 is a part for supplying power to the electrode 141 of the plasma generator device 140 in order to generate plasma discharge at the plasma generator device 140.

FIG. 2 is a drawing illustrating a spray apparatus of a hydrogen peroxide plasma generator device with a double jet nozzle according to an embodiment of the present invention.

As shown in FIG. 2, the spray apparatus 130 may be configured of the double-jet nozzle 133 disposed at the center of the spray apparatus body 130a of which the first jet nozzle 131 sprays the liquid hydrogen peroxide supplied from the hydrogen peroxide container and supplier 110 and the second jet nozzle 132 smaller than the first jet nozzle 131 in diameter is extend form the first jet nozzle 131, an air passage 134 formed to surround the double-jet nozzle 133 with disposing the double-jet nozzle 133 at the center, and a mist spray hole 135 formed at an end of the spray apparatus body 130a to expose the second jet nozzle 132 of the double jet nozzle 133 and the air passage 134.

FIG. 3 is a drawing illustrating a spray apparatus of a hydrogen peroxide plasma generator device with a double jet nozzle according to another embodiment of the present invention.

As shown in FIG. 3, the spray apparatus 130 may be configured of the double-jet nozzle 133 disposed at the center of the spray apparatus body 130a of which the first jet nozzle 131 sprays the liquid hydrogen peroxide supplied from the hydrogen peroxide container and supplier 110 and the second jet nozzle 132 holds the first jet nozzle 131 in the inside, receives air supplied from the air supplier 120 and sprays the air with the liquid hydrogen peroxide, an air passage 134 covering the double-jet nozzle 133 with disposing the double-jet nozzle 133 in the inside, and a mist spray hole 135 formed at an end of the spray apparatus body 130a to expose the second jet nozzle 132 of the double jet nozzle 133 and the air passage 134.

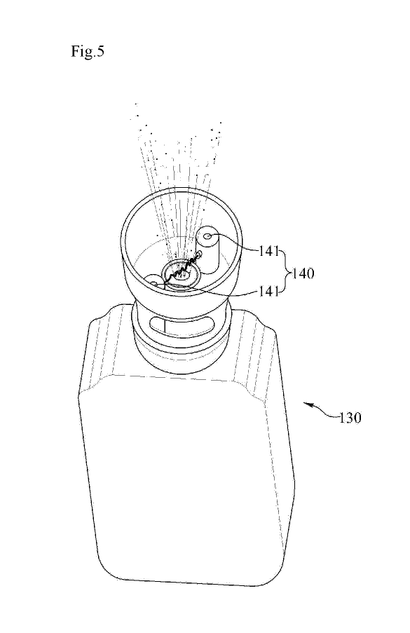

FIG. 4 is a perspective view illustrating the external appearance of the spray apparatus of a hydrogen peroxide plasma generator device with a double jet nozzle according to an embodiment of the present invention, and FIG. 5 is a drawing illustrating an operation state of passing vapor through a plasma generator device in a hydrogen peroxide plasma generator device with a double jet nozzle according to an embodiment of the present invention. FIG. 4 illustrates a schematic outer appearance of the spray apparatus 130, and FIG. 5 illustrates operation state in which hydrogen peroxide is sprayed as vapor shape from the spray apparatus 130 configured in double jet nozzle 133 and is ionized and diffused through the plasma generator device 140 installed near the spray apparatus 130.

As described above, the hydrogen peroxide plasma ionization generator device having a double-jet nozzle according to an embodiment of the present invention is configured to spray finer hydrogen peroxide and ion than hydrogen peroxide and ion sprayed from the conventional single-jet nozzle in particle size. The spray apparatus is configured in a structure of the double jet nozzle forming air inflowing passage thereby improving proper space permeation and mobility of molecule structure in order to remove efficiently microbes, bacteria and mold at a room inside and a fine space between instruments, by constructing the double-jet nozzle to spray finer particle size than the conventional single-jet nozzle. In addition, the restraint on condensation frequently occurred during sterilization and the diffusion ability for permeating in the fine space where the vapor molecule was not permeated are improved, thereby improving sterilizing effect at the room inside or the fine space between instruments.

The present invention will be changed and modified by those skilled in the art, and the scopes of the inventive concepts are to be determined by the following claims.

What is claimed is:

1. A hydrogen peroxide plasma ionization generator device 100 having a double-jet nozzle comprising:
a hydrogen peroxide container and supplier 110 containing and supplying liquid hydrogen peroxide;
an air supplier 120 with an air pump 121 receiving external air and supplying pressurized air;
a spray apparatus 130 configured in a double jet nozzle 133 of a first jet nozzle 131 for spraying vapor shaped liquid hydrogen peroxide supplied from the hydrogen peroxide container and supplier 110 and a double jet nozzle 132 spraying the vapor of the hydrogen peroxide from the first jet nozzle 131 with adding air supplied from the air supplier 120;
a plasma generator device 140 installed near the second jet nozzle 132 of the spray apparatus 130 and generating plasma discharge to ionize the hydrogen peroxide sprayed at the vapor form to ROS (Reactive Oxygen Species) and to diffuse the ROS; and
a power supplier 150 for supplying power to the electrode 141 of the plasma generator device 140 in order to generate plasma discharge at the plasma generator device 140, wherein the spray apparatus 130 is composed of the double-jet nozzle 133 disposed at the center of the spray apparatus body 130a and formed of the first jet nozzle 131 spraying the liquid hydrogen peroxide supplied from the hydrogen peroxide container and supplier 110 and the second jet nozzle 132 holding the first jet nozzle 131 in the inside, receiving air supplied from the air supplier 120 and spraying the air with the liquid hydrogen peroxide, an air passage 134 covering the double-jet nozzle 133 with disposing the double-jet nozzle 133 in the inside, and a mist spray hole 135 formed at an end of the spray apparatus body 130a to expose the second jet nozzle 132 of the double jet nozzle 133 and the air passage 134.

2. A hydrogen peroxide plasma ionization generator device having a double-jet nozzle of claim 1, wherein the hydrogen peroxide container and supplier contains and supplies liquid hydrogen peroxide, wherein the liquid hydrogen peroxide is 7.5% low concentration hydrogen peroxide which is safe in normal exposure.

3. A hydrogen peroxide plasma ionization generator device having a double-jet nozzle of claim 1 or 2, wherein the size of the hydrogen peroxide and ion particles sprayed through the double jet nozzle 133 of the spray apparatus 130 are 0.3˜3 micron.

4. A hydrogen peroxide plasma ionization generator device having a double-jet nozzle of claim 3, wherein the plasma generator device 140 generates plasma discharge of 17,000 volt such that vapor of hydrogen peroxide sprayed through the first and second jet nozzles 131 and 132 of the spray apparatus 130 is ionized to reactive oxygen species to diffuse rapidly.
